(19) United States
(12) Patent Application Publication
(10) Pub. No.: US 2024/0122515 A1
(43) Pub. Date: Apr. 18, 2024

(54) SYSTEM FOR FASTENING OPTICALLY PUMPED MAGNETOMETERS (OPM), AND ELASTOMER MATRIX WHICH INCORPORATES A SYSTEM PART INTENDED TO BE FIXED TO A MAGNETOENCEPHALOGRAPHY DEVICE

(71) Applicant: COMMISSARIAT A L'ENERGIE ATOMIQUE ET AUX ENERGIES ALTERNATIVES, Paris (FR)

(72) Inventors: Etienne LABYT, Grenoble Cedex 09 (FR); William FOURCAULT, Grenoble Cedex 09 (FR); llea PAQUIN-HONORE, Lille (FR); Guilhem LAFFONT, Lille (FR)

(73) Assignee: COMMISSARIAT A L'ENERGIE ATOMIQUE ET AUX ENERGIES ALTERNATIVES, Paris (FR)

(21) Appl. No.: 18/547,740

(22) PCT Filed: Feb. 17, 2022
(86) PCT No.: PCT/EP2022/053885

§ 371 Date: Aug. 24, 2023

(30) Foreign Application Priority Data
Feb. 25, 2021 (FR) 21 01838

(51) Int. Cl. A61B 5/245; A61B 5/00

(52) U.S. Cl. CPC A61B 5/245; A61B 5/6803; A61B 2562/0223

(57) ABSTRACT

An OPM sensor fastening system includes a support socket for positioning the sensor, the support socket having a base and a housing for accommodating a portion of the OPM sensor, and a locking part for locking the sensor in the support socket, the locking part having an open base suitable for accommodating the base of the socket, a housing for accommodating a portion of the OPM sensor, and a removable partition suitable for letting the OPM sensor pass. The locking part is configured to press-fittingly cooperate with the support socket so as to blockingly wedge the OPM sensor in the longitudinal position relative to the socket.

### TECHNICAL FIELD

The present invention relates to the general field of magnetoencephalography (MEG).

It relates more particularly to the fastening of sensors of the optically pumped magnetometer (OPM) type to an MEG device.

Although it is described with reference to an application in which the MEG device is a helmet, the MEG device may be a magnetocardiography chest belt or a fetal magnetocardiography or magnetoencephalography abdominal/pelvic belt.

### PRIOR ART

Optically pumped magnetometers (OPM) are beginning to be used in magnetoencephalography prototypes which are devices that record the magnetic field of the brain: [1], [2], [3].

These sensors are physically independent of one another and may be positioned as close as possible to the patient's scalp. The benefit of this is that it optimizes the signal-to-noise ratio because the magnetic field decreases with distance.

The importance of limiting the movements of the sensors has been demonstrated by simulation and by actual recordings: [3] and [4].

Two main sources of movement have been identified:
movements of the sensors in their support;
movements of the sensors relative to the head of the subject.

These two sources of movement generate interference in the signals and introduce bias into the methods used for locating the brain regions which have been recorded, adversely affecting the precision of the measurement.

This therefore means that the sensors need to be correctly held in their supports and that the sensors need to be correctly held on the head of the subject.

Patent JP2020151023 describes and claims a head-mounted support, a sensor fixing and a sensor of OPM type positioned facing the region of the cerebral cortex defined by the Brodmann area.

Patent CN105147289 itself describes and claims an MEG helmet made of an elastic material fastened on the head by a chin strap.

Patent application WO2020/084194A1 claims a rigid helmet system that can be adapted to suit various head sizes and that allows OPMs to be positioned in the right and left temporal regions, above the user's ears, in order to record cerebral responses to an auditory stimulus.

There is still a need to improve the systems for fastening OPM sensors to MEG helmets, notably those of the existing systems as mentioned hereinabove.

The general objective of the invention is therefore to at least partially address this need.

### SUMMARY OF THE INVENTION

In order to do this, a first subject of the invention is a fastening system for an OPM sensor, comprising:
a support socket for positioning the sensor, comprising:
a housing to house part of the OPM sensor, a base;
a locking piece for locking the sensor in the support socket and comprising:
a housing to house part of the OPM sensor,
a removable barrier designed to allow the OPM sensor to pass,
an open-ended base designed to house the base of the socket, the locking piece being designed to collaborate by force fitting with the support socket so as to immobilize the OPM sensor in a longitudinal position relative to the socket by wedging.

According to an advantageous embodiment, the support socket is a monobloc component of longitudinal axis made up of the base and of a group of flexible blades defining the housing, these blades extending longitudinally from the base.

As a preference, the blades of the support socket have a strip removed from their center.

According to one advantageous feature, the base of the support socket is of square or rectangular cross section with the number of blades being four.

According to another advantageous embodiment, the locking piece is a monobloc component of longitudinal axis made up of the base and of a group of flexible blades defining the housing and extending longitudinally from the base, the flexible blades each comprising a flexible lateral discontinuity, one of the discontinuities being designed to fit into the other of the discontinuities and to move apart from one another in order to constitute the removable barrier.

The removable barrier allows the blades of the locking piece to be parted so that the OPM sensor can be slipped inside. This also makes it possible to define an orifice through which the wiring harness of the cables of the OPM sensor passes.

As a preference, the flexible blades of the locking piece have a strip removed from their center.

According to one advantageous variant, at least part of the edges of the base of the locking piece having a strip removed from their center.

Advantageously, at least part of the blades comprising a grip portion, preferably outwardly curved.

Advantageously also, at least part of the inner edges of the base comprises an inner chamfer.

According to one advantageous feature, the base is of square or rectangular cross section with the number of blades being two.

Advantageously, the housing of the locking piece is designed to hold the cable to which the sensor is connected. This makes it possible to avoid any interference in the signals during a measurement.

As a preference, the support socket and the locking piece are made of a plastic, preferably polyamide.

Another subject of the invention is a block comprising a plurality of support sockets according to the fastening system described hereinabove.

A further subject of the invention is a matrix made of a flexible material and intended to be shaped and fixed to a magnetoencephalography helmet.

The matrix is preferably made of an elastomer, notably silicone.

A final subject of the invention is an assembly comprising a magnetoencephalography device and a matrix described hereinabove and fixed to the helmet, preferably by stitching.

The device may be a helmet made of textile material.

The helmet preferably comprises a chin strap and a tightening system at the back of the head.

Laces fixed to the matrix and to the helmet may advantageously be provided for fastening the helmet on the head of an individual.

The device may also be a magnetocardiography chest belt or a fetal magnetocardiography or magnetoencephalography abdominal/pelvic belt.

Thus, the invention essentially consists in a fastening system for fastening each OPM sensor to the MEG helmets which provides positioning with end stop and then locking in this position.

The system comprises two parts, namely a support socket and a locking piece. The socket supports the OPM sensor and the locking piece fits over the top to fix the sensor in position in its support socket and hold the cable leading out from the sensor.

The support sockets may be grouped together in a block typically made of a rigid plastic.

These sockets are positioned on a silicone matrix that covers the entirety of the head. This silicone matrix is fixed, preferably stitched, to a framework of a helmet, preferably made of textile, which has two tightening systems, a chin strap and a tightening system at the back of the head.

In addition to this, a collection of laces for tightening according to a precise pattern, and which are fixed to the elastomer, preferably silicone, matrix and to the helmet framework, allows the helmet to be adjusted to fit the patient's scalp as closely as possible and ensures that the helmet, and therefore the OPM sensors, do not move relative to the patient's head.

The assembly constitutes a helmet that is fully flexible and can be customized to suit the morphology and the size of the head, thanks to the various tightening systems.

The advantages of the invention are many and of these mention may be made of reliable customized positioning and locking for each OPM sensor with the support sockets nevertheless being grouped according to their location on the head.

The possibility of having different sizes—adults, child, infant—of textile helmet framework and of silicone matrix so as to be able to adapt to suit all categories of head size.

The configuration of the silicone matrix (the shape and number of sensors that can be positioned) can vary and can be stitched to a device other than the textile helmet framework. This may for example be a chest belt for magnetocardiography or an abdominal/pelvic belt for fetal magnetocardiography or magnetoencephalography.

The helmet framework made of textile, preferably a micro ventilated textile, may be replaced by an elastic cap for example.

Materials other than the preferred materials, silicone and polyamide-12, may also be used provided that they are nonmagnetic.

Further advantages and features will become better apparent from reading the detailed description given by way of nonlimiting illustration with reference to the following figures.

### BRIEF DESCRIPTION OF THE DRAWINGS

FIG. 1 is a perspective view of a monobloc component incorporating a number of support sockets of a fastening assembly for an OPM sensor according to the invention.

FIG. 2 is a perspective view of a monobloc component with support sockets according to a variant of the invention.

FIG. 3 is a perspective view of an OPM sensor to be fastened according to the invention.

FIG. 4A is a perspective view of a locking piece of an OPM sensor fastening assembly according to the invention.

FIG. 4B is another perspective view of the piece according to FIG. 4A.

FIG. 4C is a view in longitudinal section of the piece according to FIG. 4A.

FIG. 4D is another view in longitudinal section of the piece according to FIG. 4A.

FIG. 5 is a perspective view showing an OPM measurement sensor fastened by means of a fastening assembly according to the invention as installed on an MEG helmet.

FIG. 5A is a view in longitudinal section of FIG. 5, at the sensor in its position fixed longitudinally by the locking piece on the support socket of the fastening assembly.

FIG. 6A is a perspective view of an elastomer membrane intended to be fixed to an MEG helmet, the membrane comprising open-ended openings intended to house support sockets of the fastening assembly according to the invention.

FIG. 6B is another perspective view of FIG. 6A.

FIG. 7 is a view from above showing an arrangement of blocks incorporating support sockets as they are in the configuration installed in different zones on an MEG helmet.

FIG. 8 is a side view showing an MEG helmet placed on a head of an individual and incorporating support sockets according to the invention, also showing the line of the tightening laces used for adjusting the MEG helmet to be the closest possible fit to the scalp of the individual.

FIG. 9 is a rear view of the MEG helmet according to FIG. 8, offering another view of the path of the tightening laces used for adjusting the MEG helmet to be the closest possible fit to the scalp of the individual.

### DETAILED DESCRIPTION

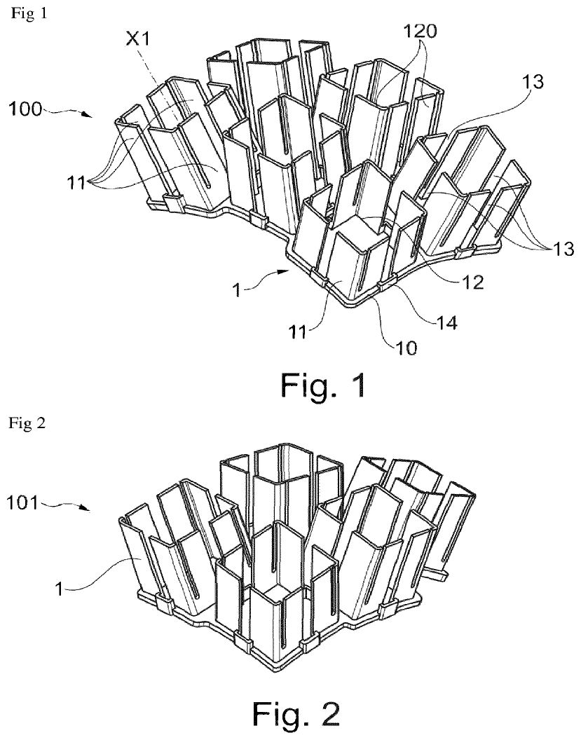

FIG. 1 shows a block 100 grouping together a number of six support sockets 1 of a fastening system according to the invention.

The block 101 shown in FIG. 2 comprises five support sockets 1. As explained hereinafter, these sockets may be grouped together in blocks of 3, 4, 5 or 6. Each block 100, 101 of sockets has a radius of curvature that is suited to the zone of the scalp that it is to cover. The number of support sockets per block varies according to the zone and according to the curvature constraints thereof.

Each of the sockets 1 is made of a rigid plastic, preferably polyamide-12, and is intended to support one OPM sensor.

In the example illustrated, a support socket 1 is a monobloc component of longitudinal axis (X1), with a square cross section complementing that of an OPM sensor.

The socket 1 consists of a base 10 and of a group 11 of flexible blades 12 defining a housing 12 for the OPM sensor. These blades 12 extend longitudinally from the base 10.

Each flexible blade 12 has a strip 13 removed from its center. This opening 13 minimizes contact with certain parts of the OPM sensors 2. These openings 13 distributed symmetrically over each of the faces of the base 10 allow an OPM sensor to be oriented in all four possible directions, namely directions at 90° from one another.

The edges of the flexible blades 12 have clearances 120 to make it easier to position the sensor in its support.

Each edge of the base 10 incorporates a contour 14 the purpose of which is to allow an element projecting from the OPM sensor, such as a pip 23, to pass. This pip 23 is created when the measurement probe of the sensor, which contains helium, is heat sealed to form the sensitive element of the sensor.

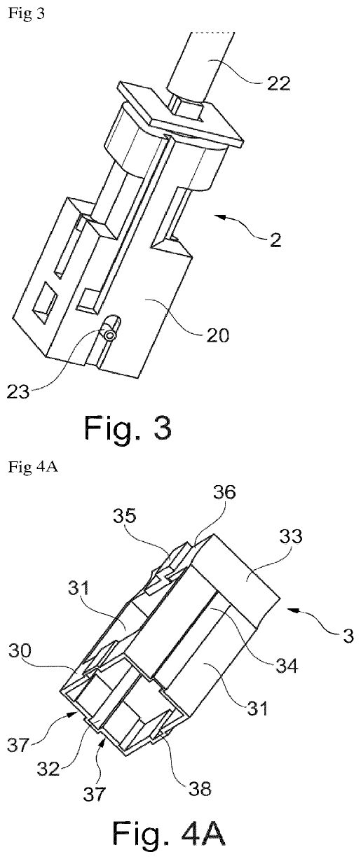

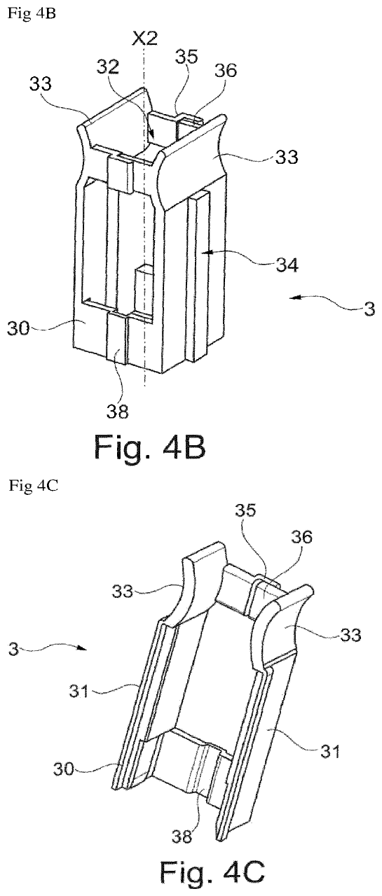

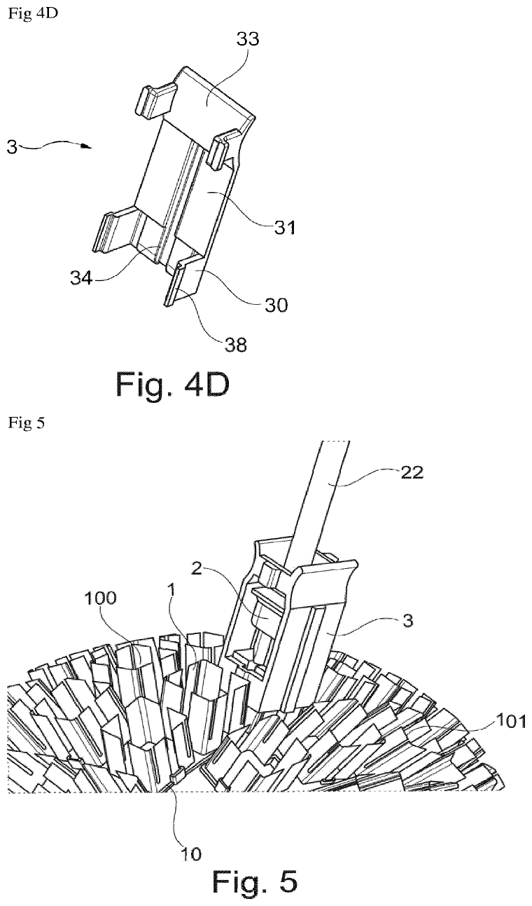

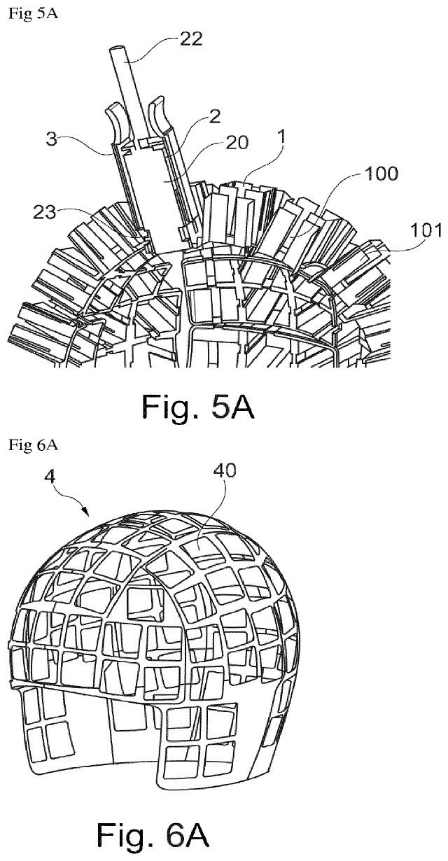

FIG. 3 shows an example of an OPM sensor to be fastened according to the invention. Such an OPM sensor 2 essentially comprises a casing 20 of square base housing the measurement probe connected to a cable 22. A pip 23 projects outwards from one of the edges of the casing 20.

The free end of the OPM sensor 2 is intended to fit as close as possible to, or even come in direct contact with, the head of a person on which a magnetoencephalography is to be performed. Thus, the head, and more specifically the scalp, constitutes the mechanical end stop for the OPM sensor 2.

The other piece of the fastening system is a locking piece 3 made of rigid plastic, preferably also of polyamide-12, which allows the OPM sensor to be locked in position in its support socket 1 by wedging.

This piece 3 is made of a base 30 extended by two flexible lateral blades 31 over the majority of the height of the piece internally delimiting a housing 32. Each blade 31 ends with a curved grip part 33 to make it easier to grasp.

The four edges of the base 30 and the two lateral blades 31 are slotted at their center 34, so as to minimize potential contact with the sensitive parts of an OPM sensor.

A tab 35, 36 is arranged perpendicular to each of the curved parts 33. Each of the tabs 35, 36 which face one another are designed to fit one into the other.

By parting these two tabs 35, 36 from one another it is possible to slip the OPM sensor into the housing 12 together with the cable 22 or a wiring harness of cables connected to the OPM sensor.

The edges of the base 30 internally have at least one chamfer 37 to facilitate the fitting of the locking piece 3 over the support socket 1 with the OPM sensor.

Each edge of the base 30 is provided with a contour 38 which serves to allow the pip 23 to pass and/or to minimize any potential contact with the sensitive parts of the OPM sensor 2.

The mounting of the fastening system on an OPM helmet and the positioning and locking of an OPM sensor are now described.

The base 10 allows the socket 1 to be held on a silicone matrix 4. The lower edge of the base 10 acts as an end stop for the silicone matrix 4.

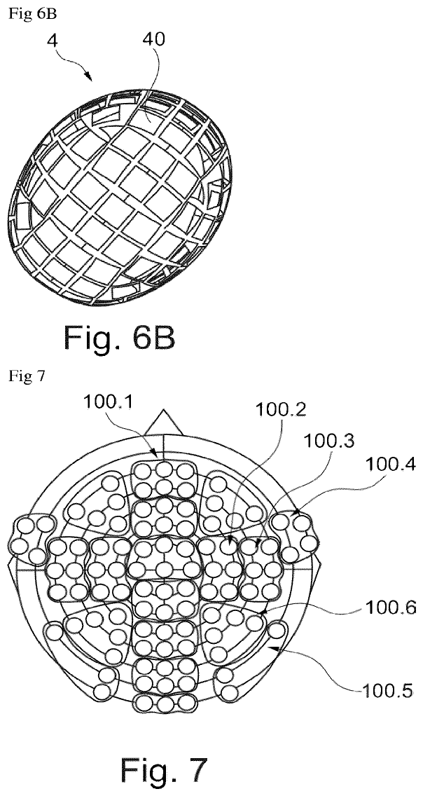

The matrix 4 is preferably made of biocompatible talced translucent 50-shore silicone. Each socket of the block is positioned in an orifice 40 provided for that purpose in the silicone matrix as shown in FIGS. 6A and 6B.

Each of the orifices 40 is of a square shape with the same dimensions as those of the base 10.

The matrix 4 is intended to cover the entirety of the surface of the head facing the cerebral regions (forehead, temples, scalp and the base of the head) as shown in FIG. 7.

This FIG. 7 shows the distribution of the orifices 40 that accept the sockets which distribution has been optimized so that the maximum number of OPM sensors can be fitted over the entirety of the surface of the head facing the regions of the brain.

The matrix 4 thus allows the fitting of up to 97 OPM sensors. The layout of this matrix 4 is based on blocks of 3 to 6 sockets so that these correspond to the various regions of the scalp. Thus, in FIG. 7, the blocks 100.1, 100.2, 100.3, 100.4, 100.5 and 100.6 correspond respectively to the median zone, the upper and lower median lateral zones, the temporal zones, the lateral occipital zones and the quadrant zones.

Once an OPM sensor 2 has been inserted through the locking piece 3, the latter is positioned on its support socket 1, as close as possible to the scalp of the head, or even so far as to be in direct contact therewith.

The locking piece 3 is lowered onto the support socket 1 and forcibly push-fitted onto same so as to lock the position of the OPM sensor by wedging it and comes into mechanical abutment with the base 10 around the flexible blades 12.

In other words, the OPM sensor 2 is inserted into the housing 12 of the support socket without mechanical abutment as such, and the locking piece 3, through the wedging effect it affords, fixes the position of the sensor 2 relative to the support socket 10.

As shown in FIGS. 6 and 6A, the removable barrier created by the tabs 35, 36 that can be parted allows the cable 22 to which the sensor 2 is connected to pass, and the housing 32 formed in the locking piece 3 advantageously holds the cable 22, making it possible to avoid any interference with the signals during a measurement.

The silicone matrix 4 is stitched to a helmet framework 5, made of micro ventilated textile.

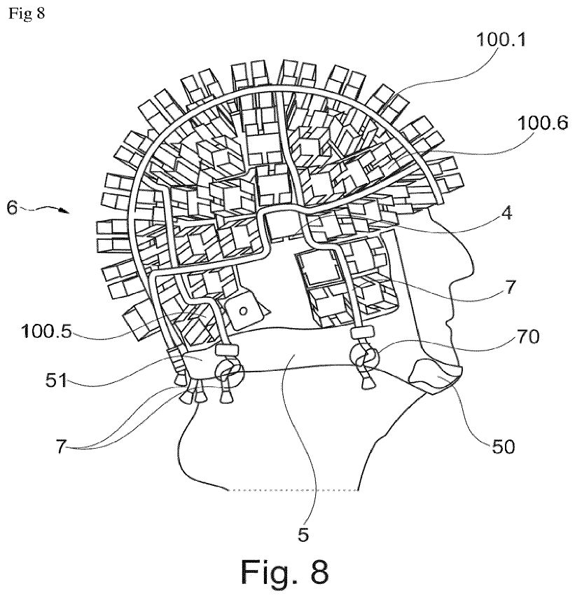

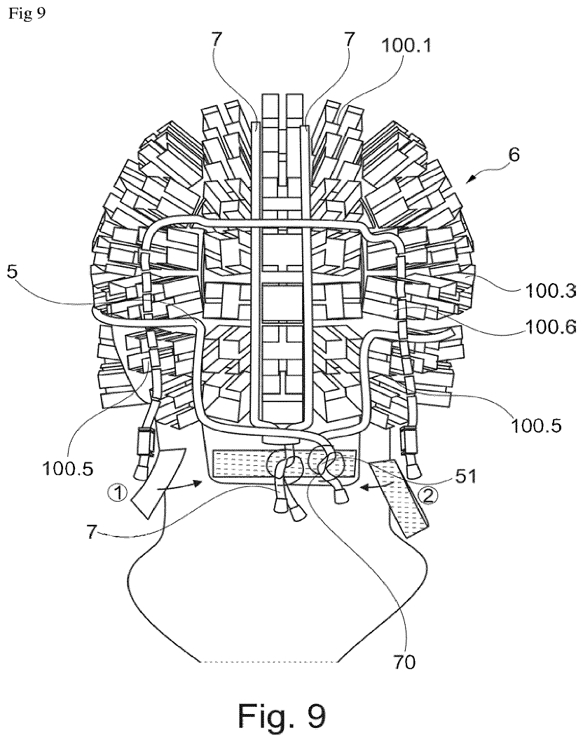

The matrix 4, the framework 5 and the blocks of support sockets 100.1, 100.2, 100.3, 100.4, 100.5 and 100.6 form a magnetoencephalography helmet 6, as illustrated in FIGS. 8 and 9.

This framework 5 has a chin strap 50 and a rear tightening system 51, of the hook and loop type, so as to adjust the helmet to the morphology of the patient's head.

Laces 7 fixed by small casings 70 to the matrix 4 and to the textile helmet framework 8 allow the OPM sensors positioned in their sockets 1 to be pressed as intimately as possible against the scalp.

Each of the tightening laces 7 preferably has an S-shaped cord-lock system without metal parts so as not to generate any magnetic interference liable to impair the operation of the OPM sensors.

The invention is not restricted to the examples that have just been described; features of the examples illustrated may notably be combined with one another into variants that have not been illustrated.

Other variants and improvements may be envisioned without thereby departing from the scope of the invention.

### LIST OF CITED REFERENCES

[1]: Boto et al., «Moving magnetoencephalography towards real-world applications with a wearable system». Nature. 2018 Mar. 29; 555(7698):657-661 DOI: 10.1038/nature26147.
[2]: Hill et al., «Multi-Channel Whole Head OPM-MEG»: Helmet design and a comparison with a conventional system NeuroImage Vol 219, 1 Oct. 2020, 116995https://doi.org/10.1016/j.neuroimage.2020.116995.
[3]: Boma et al, «Non-Invasive Functional-Brain-Imaging with an OPM-based Magnetoencephalography System» PLoS ONE 15(1). 2020 https://doi.org/10.1371/journal.pone.0227684.
[4]: Leonardo Duque-Muñoz et al. «Data-driven model optimization for optically pumped magnetometer sensor arrays». 1 Jul. 2019. https://doi.org/10.1002/hbm.24707.

1. A fastening system for an OPM sensor, comprising:
a support socket for positioning the sensor, comprising:
a base, and
a first housing to house part of the OPM sensor, and
a locking piece for locking the sensor in the support socket and comprising:
an open-ended base designed to house the base of the socket,
a second housing to house part of the OPM sensor, and
a removable barrier designed to allow the OPM sensor to pass,
the locking piece being configured to collaborate by force-fitting with the support socket so as to immobilize the OPM sensor in a longitudinal position relative to the socket by wedging.

2. The fastening system as claimed in claim 1, the support socket being a monobloc component of longitudinal axis made up of the base and of a group of flexible blades defining the first housing, the blades extending longitudinally from the base.

3. The fastening system as claimed in claim 2, the blades of the support socket having a strip removed from their center.

4. The fastening system as claimed in claim 2, the base of the support socket being of square or rectangular cross section with a number of blades being four.

5. The fastening system as claimed in claim 1, the locking piece being a monobloc component of longitudinal axis made up of the open-ended base and of a group of flexible blades defining the second housing and extending longitudinally from the open-ended base, the flexible blades each comprising a flexible lateral discontinuity, one of the discontinuities being designed to fit into another one of the discontinuities and to move apart from one another in order to constitute the removable barrier.

6. The fastening system as claimed in claim 5, the flexible blades of the locking piece having a strip removed from their center.

7. The fastening system as claimed in claim 6, at least part of edges of the base of the locking piece having a strip removed from their center.

8. The fastening system as claimed in claim 5, at least part of the blades comprising a grip portion.

9. The fastening system as claimed in claim 5, at least part of inner edges of the base of the locking piece comprising an inner chamfer.

10. The fastening system as claimed in claim 5, the base being of square or rectangular cross section with a number of blades being two.

11. The fastening system as claimed in claim 1, the second housing of the locking piece being designed to hold a cable to which the sensor is connected.

12. The fastening system as claimed in claim 1, the support socket and the locking piece being made of a plastic.

13. An assembly comprising a magnetoencephalography device and a matrix made of a flexible material that is shaped and fixed to the device the device being a framework of a helmet made from a textile material, the matrix comprising orifices in each of which a support socket of a block comprising a plurality of support sockets according to the fastening system as claimed in claim 1 is configured to be placed.

14. The assembly as claimed in claim 13, the helmet comprising a chin strap.

15. The assembly as claimed in claim 13, laces being fixed to the matrix and to the framework in order to fasten the helmet on the head of an individual.

16. An assembly comprising a magnetoencephalography device and a flexible-material matrix that is shaped and fixed to the device, the device being a magnetocardiography chest belt or a fetal magnetocardiography or magnetoencephalography abdominal/pelvic belt, the matrix comprising orifices in each of which a support socket of a block comprising a plurality of support sockets according to the fastening system as claimed in claim 1 is configured to be placed.

17. The fastening system as claimed in claim 8 wherein the grip portion is outwardly curved.

18. The fastening system as claimed in claim 12, the support socket and the locking piece being made of polyamide.

19. The assembly as claimed in claim 13, wherein the matrix is made of an elastomer.

20. The assembly as claimed in claim 16, wherein the matrix is made of an elastomer.
